(19) United States
(12) Patent Application Publication
(10) Pub. No.: US 2009/0197975 A1
(43) Pub. Date: Aug. 6, 2009

(54) COMPOSITIONS FORMULATED FOR SOLVENT-REGULATED DRUG RELEASE

(75) Inventors: Mao-Jung Maurice Lien, Maple Ridge (CA); Dean-mo Liu, Richmond (CA); Doug Smith, Vancouver (CA); Arc Rajtar, Port Moody (CA)

Correspondence Address: RISSMAN HENDRICKS & OLIVERIO, LLP, 100 Cambridge Street, Suite 2101, BOSTON, MA, 02114, US

(73) Assignee: MIV Therapeutics Inc., Vancouver (CA)

(21) Appl. No.: 12/159,952

(22) PCT Filed: Dec. 23, 2005
(86) PCT No.: PCT/CA2005/001969

§ 371 Date: Dec. 19, 2008

(51) Int. Cl. A61K 47/44; A61K 47/16; A61K 47/12

(52) U.S. Cl. 514/783; 514/788; 514/784; 514/772

(57) ABSTRACT

This application relates to a composition comprising a mixture of different organic solvents formulated for controlled drug release. The release profile of the drug can be regulated by adjusting the compositional ratios of the solvents. In one embodiment of the invention a first solvent is water-soluble and a second solvent is water-insoluble. The first and second solvents are miscible and together form a solution containing the drug. The hydrophobicity of the composition can be adjusted by altering the relative amount of the second solvent. The composition also includes a solid lipid dissolved in the drug-containing solution. In aqueous environments the lipid may precipitate to form a thin membrane in an outer surface portion of the composition, thereby further regulating the release of the drug. The membrane is preferably renewable. That is, as the outermost portion of the lipid is biodegraded at a target location in vivo, additional outer portions of the lipid precipitate to renew the thin membrane. The composition may be formulated, for example, as a suspension, nanoparticle, microparticle, paste or thin film coating. In one particular embodiment, the composition may be applied to an implantable medical device, such as a cardiovascular stent.

### TECHNICAL FIELD

This application relates to compositions formulated for controlled drug release.

### BACKGROUND

Oil-based drug delivery systems are well known in the prior art and can be divided generally into either liquid-phase or solid-phase delivery systems. Solid-phase delivery systems typically involve dispersal or dissolution of a drug in a melted lipid or combination of lipids. The lipid is then shaped in specific dosage forms such as solid lipid nanoparticles or microparticles (R H Muller et al. “Solid lipid nanoparticles for controlled drug delivery-a review of the state of the art”, European Journal of Pharmaceutics and Biopharmaceutics, 50, pp. 161-177 (2000), R H Muller et al., Pharm. Ind., 61, pp. 174-178 (1999), D. Hou et al., “The production and characteristics of solid lipid nanoparticles”, Biomaterials, 24, pp. 1781-1785 (2003), M. Stuchlik et al., “Lipid-based vehicle for oral drug delivery”, Biomed. Papers., 145[2], pp. 17-26 (2001)). In many cases polymer must be added to the solid-phase compositions to enable the stable formation of solid particulate forms.

Liquid-phase delivery systems typically employ solubilizing excipients for direct and/or sustained release of oral and injection formulations. As reviewed by Robert S. Strickley in “Solubilizing Excipients in Oral and Injectable Formulations”, Pharmaceutical Research., 21[2], pp. 201-230, 2004, most existing drug formulations using solubilizing agents include water-soluble organic solvents, non-ionic surfactants, water-insoluble lipids, organic liquid/semi-solids, or various cyclodextrins/phospholipids. Some prior art approaches employ a mixture of water-based (aqueous) solutions containing water, ethyl alcohol and polyethylene glycol. Other formulations employ a co-solvent system suitable for injection administration. However, conventional liquid-phase formulations are not specifically adapted for regulating the rate of drug release by adjusting the relative compositional ratio of both hydrophilic and hydrophobic solvents and lipid additives. This is particularly the case in respect of the targeted delivery of water-insoluble drugs to aqueous environments in vivo.

The need has therefore arisen for improved compositions and methods for regulating drug release.

### SUMMARY OF INVENTION

In accordance with the invention, a composition is described comprising a solution formed from a water-soluble first organic solvent and a water-insoluble second organic solvent, wherein the first and second solvents are miscible. The composition further comprises at least one therapeutic agent and at least one lipid dissolved in the solution. The compositional ratios of the first and second solvents regulate the rate of release of the therapeutic agent from the composition. For example, the hydrophobicity of the composition can be adjusted by altering the relative amount of the second solvent.

The invention also relates to a method of formulating a composition comprising a therapeutic agent. The method comprises the steps of (a) providing a first water-soluble organic solvent; (b) dissolving at least one therapeutic agent in the first solvent to form a first solution; (c) adding a second water-insoluble organic solvent to the first solution, wherein the first and second solvents are miscible to form a second solution containing the therapeutic agent; (d) dissolving a lipid in the first solvent to form a third solution; and (e) adding the third solution to the second solution to form the composition.

The invention also relates to the use of a composition formulated in accordance with the invention at a target location. The use may comprise delivering the composition to the target location and allowing the therapeutic agent to elute at the target location at a rate dependent upon the relative concentrations of the first and second solvents in the composition. The composition may be formulated, for example, as a suspension, nanoparticle, microparticle, paste or thin film coating. In one particular embodiment, the composition may be applied to an implantable medical device, such as a stent. The target location may be an aqueous environment, such as blood or body tissues. In one particular embodiment the therapeutic agent may be a hydrophobic drug which is released at a controlled rate in the aqueous environment.

The lipid component of the composition may ordinarily be in a solid form at temperatures below about 40° C. In aqueous environments the lipid may precipitate to form a thin membrane in an outer surface portion of the composition, thereby further regulating the release of the therapeutic agent. The membrane is preferably renewable. That is, as the outermost portion of the lipid is biodegraded at the target location, additional outer portions of the lipid precipitate to renew the thin membrane.

### BRIEF DESCRIPTION OF DRAWINGS

In drawings which illustrate embodiments of the invention, but which should not be construed as restricting the spirit or scope of the invention in any way,

FIG. 1 is a schematic view of a method for formulating a drug composition in accordance with the invention.

FIG. 2 is a series of three photographs showing two model drug compositions formulated in accordance with the invention immersed in PBS solution. The left-hand composition comprises a combination of 40% DENA solvent and 60% castor oil solvent. The right-hand composition comprises a combination of 60% DENA solvent and 40% castor oil solvent. The pigment FeCl3 was used as a model drug for release. The top photograph shows pigment release after one minute; the middle photo-graph shows pigment release after 20 minutes; and the bottom photograph show pigment release after 24 hours.

FIG. 3 is two photographs comparing three drug compositions formulated in accordance with the invention. Each composition comprises the drug paclitaxel formulated with different compositional ratios of solvents, namely a first solvent comprising a mixture of 70% DENA and 30% DMSO and a second solvent comprising 70% castor oil and 30% soybean oil. The left-hand composition comprises 70% of the first solvent and 30% of the second solvent; the middle composition comprises 60% of the first solvent and 40% of the second solvent; and the right-hand composition comprises 50% of the first solvent and 50% of the second solvent. The top photograph shows the three drug compositions after 1 minute of immersion in PBS and the bottom photograph shows the same compositions after 72 hours of immersion in PBS.

FIG. 4 is two photographs showing three drug compositions formulated in accordance with the invention containing the solid lipid stearic acid. The left-hand composition comprises 3 weight percent stearic acid; the middle composition comprises 1.5 weight percent stearic acid; and the right- and composition comprises 0.7 weight percent stearic acid. Each composition also comprises 5 weight percent of the drug paclitaxel and 3-5 weight percent FeCl3 as an indicator of drug elution. The top photograph shows the three drug compositions after 1 minute of immersion in PBS and the bottom photograph shows the same compositions after 150 hours of immersion in PBS.

FIG. 5 is a schematic view illustrating controlled release of a drug from a composition formulated in accordance with the invention.

FIG. 6 is a series of three photographs showing three compositions formulated in accordance with the invention having varying solid lipid concentrations. Each composition comprises 10 weight percent paclitaxel formulated with a mixture of a first solvent comprising DENA and DMA and a second solvent comprising castor oil and oleic acid. The top photograph shows a composition containing no stearic acid and having a compositional ratio of 50% of the first solvent and 50% of the second solvent; the middle photograph show a solid-like, transparent gel containing 8% stearic acid and having a compositional ratio of 80% of the first solvent and 20% of the second solvent; and the bottom photograph shows a solid-like opaque gel containing 8% stearic acid and having a compositional ratio of 50% of the first solvent and 50% of the second solvent.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

This application relates to compositions comprising a mixture of different solvents formulated for controlled drug release. As described below, the release profile of the drug can be regulated by adjusting the compositional ratios of the solvents.

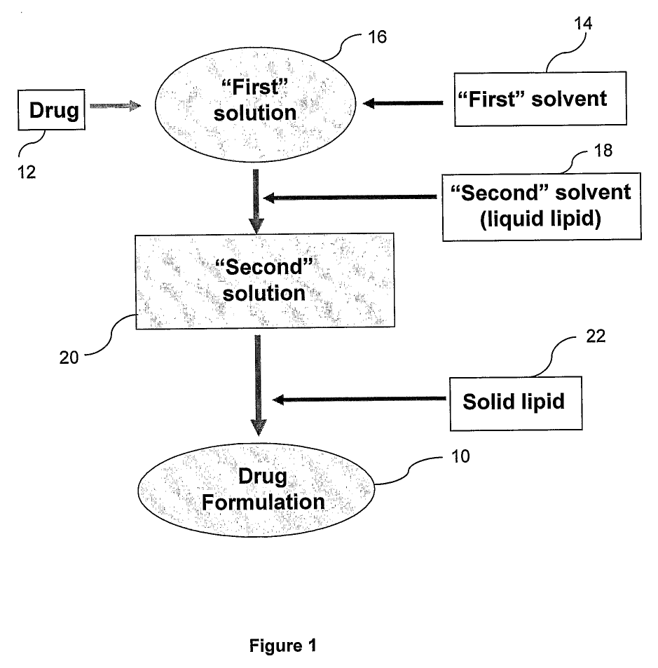

FIG. 1 is a schematic view of a method for formulating a drug composition 10 in accordance with the invention. The composition 10 includes a therapeutic agent, such as a drug 12, which is dissolved in a first solvent 14 to form a first solution 16. Drug 12 may be either water-soluble or water-insoluble. Examples of some important water-insoluble drugs include anti-cancer drugs, anti-inflammatory drugs and anti-immunosuppressant drugs and the like. First solvent 14 is a water-soluble organic solvent such as, but not limited to, dimethyl sulfoxide (DMSO), N,N-diethylnicotinamide (DENA), N,N-dimethylformamide (DMF), N,N-dimethylacetamide (DMA), Cremophor EL and their derivatives. The concentration of drug 12 in first solvent 14 may vary between about 0.001 and 70 weight percent.

After the drug-containing first solution 16 is prepared, a second solvent 18 is slowly added to solution 16 (FIG. 1). The second solvent is a water-insoluble liquid lipid such as, but not limited to, soybean oil and its derivatives, castor oil, oleic oil, peppermint oil and vegetable oils. The first and second solvents 14, 18 are miscible and combine to form a second drug-containing solution 20. The concentration of second solvent 18 in the second solution 20 may vary widely between about 5 to 85 weight percent, or more preferably between about 10 to 60 weight percent.

The final step in the formulation method is to add a solid lipid 22 to second solution 20. Lipid 22 may be added directly to solution 20 or more preferably may first be dissolved in first solvent 14. In the latter case, a third solution (not shown) is formed which is then added to second solution 20. Preferably lipid 22 is selected so that it is soluble in first solvent 14, but is non-soluble in second solvent 18. Lipid 22 is preferably a solid at temperatures below about 40° C. In one embodiment lipid 22 is a small molecule hydrocarbon of between about 12-24 carbons in length. Lipid 22 is preferably biocompatible and biodegradable in the human body and can be removed enzymatically or by other metabolic mechanisms. Examples of suitable solid lipids 22 include, but are not limited to, stearic acid, beeswax, 12-hydroxyl stearic acid, glycerol behenate, Compritol™ and hydrogenated castor oil. The concentration of lipid 22 in the final drug composition 10 may vary between a range of from 0 to 20 weight percent, or preferably from 0.01 to 10 weight percent, or more preferably, from 0.05 to 5 weight percent.

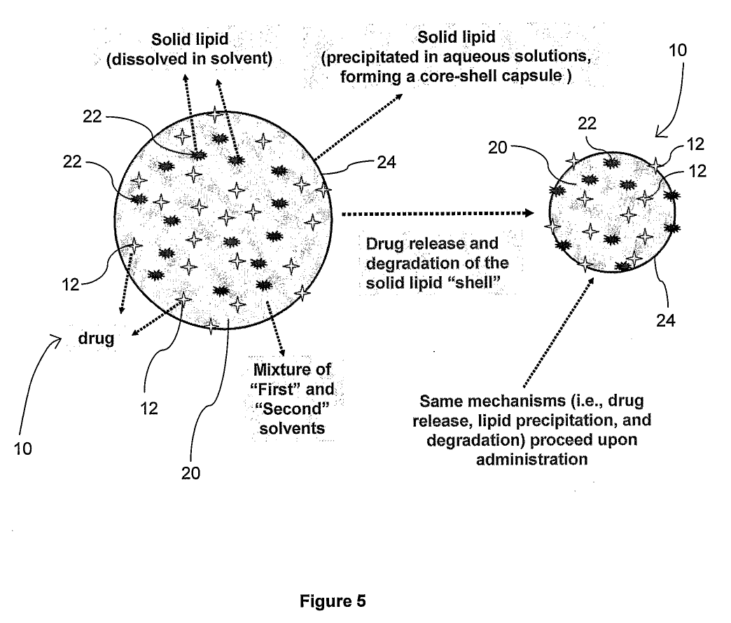

The inclusion of lipid 22 in composition 10 is advantageous for several reasons. For example, lipid 22 helps to stabilize droplets of composition 10, such as nano-scale size droplets produced by an emulsification process. Further, lipid 22 provides a thin, solid molecular layer on the outer surface of composition 10 when composition 10 is exposed to an aqueous environment, such as blood and body fluids. This feature if best shown in FIG. 5 and is described further below.

The formulation of the drug composition 10 according to the method of FIG. 1 can be carried out in ambient conditions. No particular environmental controls, such as atmosphere, moisture, oxygen partial pressure, ambient light or the like are of critical concern, except in the case if vulnerable active agents are used.

The resulting drug composition 10 is biodegradable, biocompatible and polymer-free. Depending upon the relative ratio of the constituent ingredients, composition 10 is designed to be a homogeneous oily phase from a highly-viscous liquid to a solid gel. The specific gravity of composition 10 may vary within a range of about 0.90 to 1.15. Composition 10 may be formulated in various dosage forms including suspensions, emulsions, capsules, nanoparticles, microparticles, pastes or thin film coatings. In the case of thin-layer dosage forms such as coatings, a specific gravity greater than 1.00 is preferable whereas for other applications a specific gravity below 1.00 is desirable.

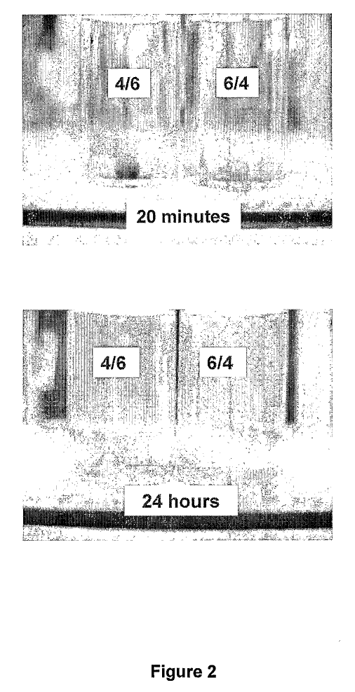

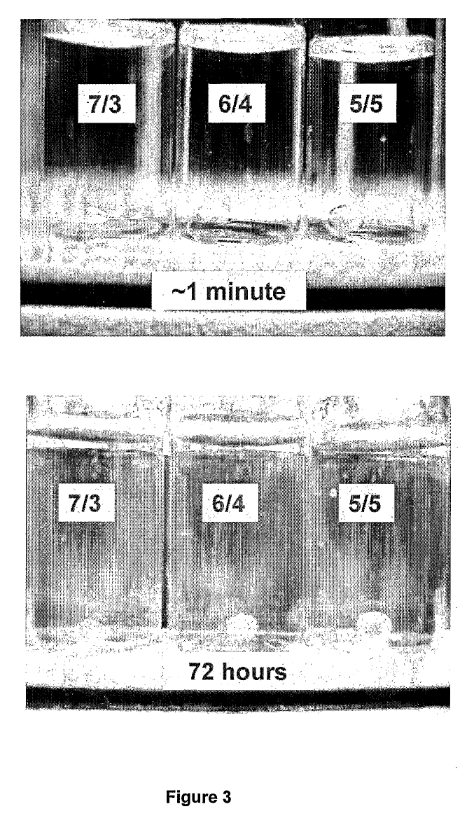

An important feature of the invention is that the release profile of drug 12 from composition 10 may be controlled by adjusting the relative amounts of the first and second solvents 14, 18. That is, the hydrophobicity of composition 10 is “tunable” by varying the relative amount of the water-insoluble second solvent 18. For example, the higher the relative concentration of second solvent 18, the slower drug 12 will be released. This feature is illustrated in FIGS. 2 and 3.

FIG. 2 is a series of three photographs showing two model drug compositions 10 formulated in accordance with the invention immersed in PBS solution. The left-hand composition comprises a combination of 40% DENA (first solvent 14) and 60% castor oil (second solvent 18). The right-hand composition comprises a combination of 60% DENA and 40% castor oil. The pigment FeCl3 was used as a model drug to monitor time-dependent release characteristics. The top photograph shows pigment release after one minute; the middle photograph shows pigment release after 20 minutes; and the bottom photograph show pigment release after 24 hours.

A spherical droplet of composition 10 was formed immediately after it was dropped into PBS, and the shape remained identical for the 24 hour drug release test. The droplets prepared for this example had a specific gravity of about 1.03-1.05, higher than PBS, and therefore causing them to settle in the bottom of the vial during the test time period. It is clearly apparent that the colored pigment (i.e. FeCl3) released quickly from the droplet with a 6/4 composition (right-hand side of FIG. 2) and the size of the 6/4 droplet shrank at a rapid rate. This suggests higher concentration of the first solvent 14 promotes a rapid drug release rate, i.e. a bursting release profile can be expected. By contrast, the 4/6 composition (left-hand side of FIG. 2), having a higher degree of hydrophobicity due to a higher concentration of the hydrophobic second solvent 18, initially exhibited a much slower rate of release than 6/4 composition, indicating the elimination or reduction of the initial bursting effect.

Further, the droplet size of the 6/4 composition showed a much lower rate of shrinkage, comparing to the 4/6 droplet (FIG. 2). The shrinkage of the droplets is solely due to a mass diffusion of the water-soluble first solvent 14 toward environment as a result of the concentration potential (i.e. mass gradient) existing between the interior of the droplet and the environment. A higher concentration of the second solvent 18 appears to inhibit or reduce considerably the outward diffusion of the first solvent 14, resulting in a slower release rate. This is likely due to a reduction of contact area between the water-soluble solvent molecules, i.e., first solvent 14, and the environmental water.

The formation of a droplet when composition 10 is exposed to an aqueous environment illustrates its hydrophobic nature and demonstrates the feasibility of forming emulsions comprising composition 10. As discussed further below, such emulsions could have various clinical applications such as suspensions for oral administration or topical use for skin wounds.

As should be apparent from the above examples, the relative proportions of the first and second solvents 14, 18 may vary depending upon the desired release profile of drug 12. For example, the compositional ratio of the first solvent 14 to the second solvent 18 in weight percent may vary between about 3:7 to 8:2, or more preferably between about 4:6 to 6:4.

In the example of FIG. 3, the release profile of a composition 10 comprising the water-insoluble lipophilic drug paclitaxel was investigated. Paclitaxel is a well-known chemotherapy agent. FIG. 3 compares three drug compositions comprising paclitaxel formulated with different compositional ratios of first and second solvents 14, 18. The concentration of paclitaxel is maintained at 20 weight percent in first solvent 14. In this example, first solvent 14 comprises a mixture of 70% DENA and 30% DMSO and second solvent 18 comprises 70% castor oil and 30% soybean oil. With reference to FIG. 3, the left-hand composition 10 comprises 70% of first solvent 14 and 30% of second solvent 18 (7/3); the middle composition comprises 60% of first solvent 14 and 40% of second solvent 18 (6/4); and the right-hand composition comprises 50% of first solvent 14 and 50% of second solvent 18 (5/5). The top photograph shows the three drug compositions after 1 minute of immersion in PBS and the bottom photograph shows the same compositions after 72 hours of immersion in PBS.

With reference to FIG. 3, the appearance of a droplet of composition 10 (with a density heavier than that of PBS) is optically clear indicating the paclitaxel is completely dissolved and mutually miscible in solution 20 comprising solvents 14, 18. After 72 hours of observation, the appearance of the droplets changes considerably in both size and color. All the droplets turned from a transparent to a light white appearance, which is suggestive of some precipitation of the paclitaxel when it contacted with environmental water. However, another possible explanation is surface complexation between the castor oil and water molecules, which has been reported in the literature. The size of the droplets shrank considerably for the 7/3 composition. A lesser degree of shrinking occurred for the 6/4 and 5/5 compositions. This is similar to the FIG. 2 results due to mass diffusion of the first solvent 14 from the droplet of composition 10 to the aqueous environment. The outward diffusion of the paclitaxel-containing first solvent 14 is accompanied by the release to the environment of paclitaxel drug, which is believed to dissolve in the PBS in the presence of both DENA and DMSO, both acting as solubilizing agents. No white precipitate of paclitaxel can be visually detected in the PBS.

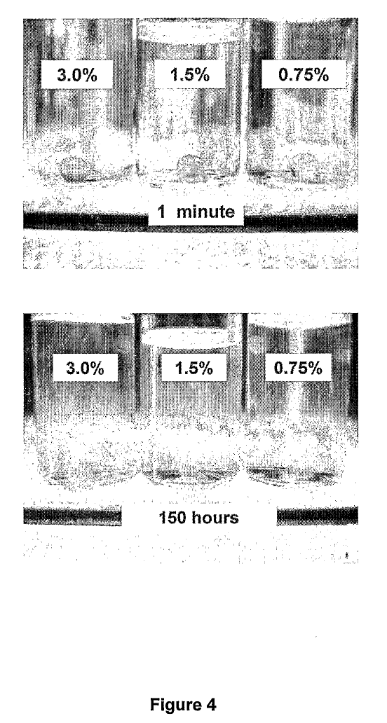

FIG. 4 shows gradual degradation of an embodiment of composition 10 comprising lipid 22. In this example, the selected solid lipid is stearic acid. FIG. 4 shows test results for three different drug compositions 10. The left-hand composition comprises weight percent stearic acid; the middle composition comprises 1.5 weight percent stearic acid; and the right- and composition comprises 0.75 weight percent stearic acid. Each composition also comprises 5 weight percent of the drug paclitaxel and 3-5 weight percent FeCl3 as an indicator of drug elution. Each composition 10 comprises a drug containing solution comprising 50% of first solvent 14 and 50% of second solvent 18 as described above (5/5). The top photograph shows the three drug compositions 10 after 1 minute of immersion in PBS and the bottom photograph shows the same compositions 10 after 150 hours of immersion in PBS.

After 150 hours of testing, the volume of the droplets of composition 10 was reduced by about 15-20%, depending on the concentration of the stearic acid (FIG. 4). The higher the concentration of stearic acid, the less the extent of droplet size reduction. Further, the appearance of the droplets became correspondingly lighter than first prepared depending upon the concentration of the stearic acid (i.e. the colour was more pronounced in the vials containing lower concentrations of stearic acid and least pronounced in the vial having a higher concentration of stearic acid, suggesting that presence of stearic acid inhibited drug release). It can be concluded that the presence of stearic acid does provide a molecularly-thin barrier layer on the droplet surface when contacting with an aqueous environment, thus regulating the mass diffusion of first solvent 14 and drug 12 (in this example paclitaxel and FeCl3) from composition 10.

This feature is illustrated diagrammatically in FIG. 5. The left-hand side of FIG. 5 shows a composition 10 comprising a drug 12 dissolved in a solution 20. Solution 20 comprises a mixture of solvents 14 and 18. Solid lipid 22 is also dissolved in solution 20. When composition 10 is exposed to an aqueous environment, lipid 22 located in outer portions of composition 10 precipitates to form a thin outer membrane 24 on the outer surface of composition 10. Membrane 24 acts as filter regulating the passage of drug 12 from the interior of composition 10 to the external environment, such as an in vivo target location. Drugs having different molecular sizes may therefore diffuse through membrane 24 at different rates. Membrane 24 is also is preferably renewable. That is, as the outermost portion of lipid 22 is biodegraded at the target location, additional outer portions of lipid 22 precipitate to renew the thin membrane 24. Thus a thin outer membrane 24 is maintained while the size of composition 10 gradually shrinks (FIG. 5). As will be appreciated by a person skilled in the art, lipid 22 may be metabolized by enzymatic activity. However, the “core-shell” structure of composition 10 is maintained.

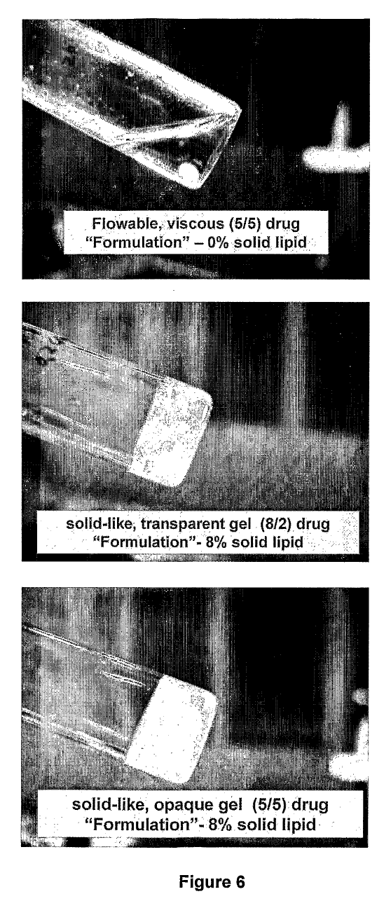

Adjusting the concentration of lipid 22 may also alter the viscosity and flowability of composition 10 as shown in FIG. 6. FIG. 6 is a series of three photographs showing three compositions 10 having varying solid lipid concentrations. Each composition comprises 10 weight percent paclitaxel formulated with a mixture of a first solvent 14 comprising DENA and DMA and a second solvent 18 comprising castor oil and oleic acid. The top photograph shows a composition containing no stearic acid and having a compositional ratio of 50% of the first solvent and 50% of the second solvent; the middle photograph show a solid-like, transparent gel containing 8% stearic acid and having a compositional ratio of 80% of the first solvent and 20% of the second solvent (8/2); and the bottom photograph shows a solid-like opaque gel containing 8% stearic acid and having a compositional ratio of 50% of the first solvent and 50% of the second solvent (5/5). In this example, the stearic acid is added to the drug solution 20 in the presence of a co-solvent, such as tetrahydrofuran (THF), which is thereafter removed by natural evaporation.

FIG. 6 demonstrates that with increasing concentration of lipid 22, composition 10 may be gelled into solid or solid-like materials. As indicated above, in the absence of stearic acid composition 10 is flowable and highly viscous. In the case of 8% stearic acid and 8/2 solvent composition a transparent solid gel is formed. In the case of 8% stearic acid and 5/5 solvent composition a white solid gel is formed. The white appearance suggests precipitation of the stearic acid used in this example. However, the stearic acid may form a network with DENA and/or DMA wherein a resulting transparent solid gel can be synthesized.

The example of FIG. 6, taken together with the example of FIG. 4, suggests multiple advantages may be achieved by adding a small amount of solid lipid 22 to composition 10. That is, lipid 22 both regulates the release of drug 12 from composition 10 and also enables formulation of composition 10 in a solid or semi-solid form suitable for coating or impregnation dosage applications. For example, composition 10 may be coated onto the surface of, or impregnated within holes or cavities of, biodegradable or non-biodegradable implantable medical devices. Many other pre-designed clinically-desirable medication forms may be envisaged by a person skilled in the art.

In a particular application of the invention, composition 10 may be formed as solidified nano-particulate or micro-particulate drug systems for drug delivery purposes. Such particulates may be formed by processes well known in the art, such as emulsification-solvent evaporation. For example, the inventors have carried out such as process using a composition 10 having a 5/5 compositional ratio of first solvent 14 and second solvent 18, 10 weight percent of paclitaxel and 8 weight percent of stearic acid (i.e. as illustrated in FIG. 6). In this example the paclitaxel was first diluted with diethyl ether as a co-solvent for use together with first solvent 14. Nanoparticulates of composition 10 were prepared by conventional emulsification synthesis using a homogenizer at a speed of 20,000 rpm for 2 minutes. The resulting emulsified phase was stable and no sign of growth of the nanoparticles was visually observed. The nanoparticles can be removed by filtration or can be used directly in presence of an aqueous solution such as saline, i.e., as an emulsion dosage form for practical medical uses.

Many other dosages forms and methods of administration of composition 10 will be apparent to a person skilled in the art.

In summary, the formulation of composition 10 provides numerous advantages over conventional formulations. The combination of mutually miscible water-insoluble and water-soluble organic solvents, and the inclusion of a small amount of dissolved solid lipid, provides benefits not achievable by prior art solid-phase and liquid-phase drug delivery systems. In particular, composition 10:
(1) is suitable for drugs that are water-soluble or water-insoluble;
(2) enables controlled delivery of drugs with a release profile ranging from pulsatile, i.e., bursting, to slow delivery;
(3) enables adjustment of hydrophobicity for different clinical needs;
(4) may be formulated as a cocktail therapy for drugs of different degrees of water solubility; and
(5) enhances the bioavailability of drugs, especially for those water-insoluble drugs.

The provision of a thin solid lipid outer membrane confers several particular advantages, such as:
(1) stabilization of composition 10 during manufacturing processes in the presence of water, such as emulsifications, to enable formation of micro-droplets or nano-suspensions;
(2) drug release can be controlled for long-term medical applications;
(3) solid lipid layer will remain as a molecular barrier layer on the droplet surface in the course of degradation process in the human body, i.e., a renewable solid lipid surface will develop on droplet surface at any time period while drug is releasing and lipid is degrading at the site of administration in vivo;
(4) drugs with different molecular size can have different release rates due to different rates of diffusion through the thin solid barrier layer on the droplet surface; and
(5) drug-eluting coating applications are feasible, wherein a polymer-free, slow release coating, for instance, on cardiovascular stents, can be easily produced.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

1. A medical device, comprising a composition applied to the device, the composition comprising:
(a) a solution comprising a water-soluble first organic solvent and a water-insoluble second organic solvent, wherein said first and second solvents are miscible;
(b) at least one therapeutic agent dissolved in said solution; and
(c) at least one lipid dissolved in said solution.

2. The device as defined in claim 1, wherein said lipid is a solid at temperatures below about 40° C.

3. The device as defined in claim 2, wherein said lipid in an outer portion of the coating precipitates when said composition is in an aqueous environment to form a membrane on an outer surface of said coating.

4. The device as defined in claim 2, wherein said composition is in the form of a gel.

5. The device as defined in claim 1, wherein said therapeutic agent is water-insoluble.

6. The device as defined in claim 1, wherein said therapeutic agent is water-soluble.

7. The device as defined in claim 5, wherein said water-insoluble therapeutic agent is dissolved in said first solvent.

8. The device as defined in claim 1, wherein said first solvent is selected from the group consisting of dimethyl sulfoxide (DMSO), N,N-diethylnicotinamllide (DENA), N,N-dimethylformamide (DMF), N,N-dimethylacetamide (DMA), polyoxyethylated castor oils and derivatives.

9. The device as defined in claim 1, wherein said second solvent is selected from the group consisting of soybean oil and its derivatives, castor oil, oleic acid, peppermint oil and vegetable oils.

10. The device as defined in claim 1, wherein the concentration of said first solvent in said composition is between about 0.001 to 70% by weight.

11. The device as defined in claim 1, wherein the concentration of said second solvent in said composition is between about 5-85% by weight.

12. (canceled)

13. The device as defined in claim 1, wherein said lipid is a small molecule hydrocarbon of between about 12-24 carbons in length.

14. The device as defined in claim 1, wherein said lipid is selected from the group consisting of stearic acid, beeswax, 12-hydroxystearic acid, glycerol behenate, hydrogenated castor oil, phospholipids and soybean lecithin.

15. The device as defined in claim 1, wherein the concentration of said lipid in said composition is within the range of about 0.05-5% by weight.

16. The device as defined in claim 1, comprising a plurality of therapeutic agents having varying degrees of water solubility.

17. The device as defined in claim 3, wherein said membrane regulates the rate of diffusion of said therapeutic agent from said composition.

18. The device as defined in claim 1, wherein the ratio of said first solvent relative to said second solvent is between about 3:7 to 8:2.

19. The device as defined in claim 18, wherein the ratio of said first solvent relative to said second solvent is between about 4:6 to 6:4.

20. The device as defined in claim 1, wherein said composition has a specific gravity between about 0.90-1.15.

21. A method of formulating a composition comprising a therapeutic agent comprising:
(a) providing a first water-soluble organic solvent;
(b) dissolving at least one therapeutic agent in said first solvent to form a first solution;
(c) adding a second water-insoluble organic solvent to said first solution, wherein said first and second solvents are miscible to form a second solution; and
(d) dissolving a lipid in said first solvent to form a third solution.

22. The method as defined in claim 21, wherein said therapeutic agent is water-insoluble.

23. The method as defined in claim 2, wherein said therapeutic agent is water-soluble.

24. (canceled)

25. The method as defined in claim 21, wherein said lipid is a solid at temperatures below about 40° C.

26. The method as defined in claim 21, wherein said method occurs at ambient temperature and pressure.

27. The method as defined in claim 21, wherein said first organic solvent is selected from the group consisting of DMSO, DENA, DMF, DMA, polyoxyethylated castor oils and derivatives.

28. The method as defined in claim 21, wherein said second organic solvent is selected from the group consisting of soybean oil and its derivatives, castor oil, oleic acid, peppermint oil, and vegetable oils.

29. The method as defined in claim 21, wherein said second solvent is added until its concentration in said composition is within the range of about 5-85% by weight.

30. The method as defined in claim 29, wherein said second solvent is added until its concentration in said composition is within the range of about 10-60% by weight.

31. The method as defined in claim 21, wherein said lipid is added until its concentration in said composition is within the range of about 0.05-5% by-weight.

32. The method as defined in claim 21, wherein said lipid is a small molecule hydrocarbon of between about 12-24 carbons in length.

33. The method as defined in claim 21, wherein said lipid is selected from the group consisting of stearic acid, beeswax, 12-hydroxystearic acid, glycerol behenate, hydrogenated castor oil, phospholipids and soybean lecithin.

34. The method as defined in claim 21, wherein said second solvent is added until the ratio of said first solvent relative to said second solvent is between about 3:7 to 8:2.

35. The method as defined in claim 34, wherein said second solvent is added until the ratio of said first solvent relative to said second solvent is between about 4:6 to 6:4.

36. A method, comprising:
(a) delivering a device as defined in claim 1 to a target location; and
(b) allowing said therapeutic agent to elute at said target location at a rate dependent on the concentration of said second solvent in said composition.

37. The method as defined in claim 36, wherein said step of delivering said composition to a target location comprises administering said composition to a subject in need of therapy in a form selected from the group consisting of a suspension, a nanoparticle, a microparticle, a paste and a thin film coating.

38. The method as defined in claim 37, wherein said administering is by a method selected from the group consisting of oral ingestion, injection, inhalation and topical administration.

39. A method, comprising:
(a) delivering a device as defined in claim 3 to a target location; and
(b) allowing said therapeutic agent to elute at said target location at a rate dependent on the concentration of said second solvent and said lipid in said composition.

40. The method as defined in claim 39, wherein said target location is an aqueous environment and wherein lipid forms a thin membrane on the outer surface of said composition at said target location, said membrane regulating the rate of elution of said therapeutic agent from said composition.

41. The method as defined in claim 40, wherein, as an outermost portion of said lipid is biodegraded at said target location, additional outer portions of said lipid precipitate to maintain said membrane.

42. A medical device, comprising a composition applied to the device, the composition for use in an aqueous environment at a target location comprising:
(a) a solution comprising a water-soluble first organic solvent and a water-insoluble second organic solvent, wherein said first and second solvents are miscible;
(b) at least one therapeutic agent dissolved in said solution; and
(c) a lipid in a solid form at temperatures below 40° C., wherein said lipid is dissolved in said solution and wherein outer portions of said lipid precipitate in said aqueous environment to form a thin renewable membrane filter regulating the elution of said therapeutic agent at said target location.

43. The composition device as defined in claim 42, wherein, as an outermost portion of said lipid is biodegraded at said target location, additional outer portions of said lipid precipitate to renew said membrane filter.

44. The device as defined in claim 1, wherein said lipid is soluble in said first solvent and insoluble in said second solvent.

45. The device as defined in claim 5, wherein said water-insoluble therapeutic agent is dissolved in a solution comprising a mixture of said first and second solvents

46. (canceled)

47. The device as defined in claim 1, wherein the device is a stent.

48. The method as defined in claim 21, wherein the device is a stent.

49. The method as defined in claim 21, further comprising (e) adding said third solution to said second solution to form said composition.
